(19) United States
(12) Patent Application Publication
(10) Pub. No.: US 2008/0305513 A1
(43) Pub. Date: Dec. 11, 2008

(54) METHOD OF MEASURING CANCER AGGRESSIVENESS AND DETECTION THEREOF

(76) Inventors: IGOR IVAKHNENKO, SILVER SPRING, MD (US); ALEXANDER BALYURA, MOSCOW (RU)

Correspondence Address: ROSENBERG, KLEIN & LEE, 3458 ELLICOTT CENTER DRIVE-SUITE 101, ELLICOTT CITY, MD, 21043, US

(21) Appl. No.: 12/194,163

(22) Filed: Aug. 19, 2008

Related U.S. Application Data
(63) Continuation-in-part of application No. 11/138,334, filed on May 27, 2005.

(30) Foreign Application Priority Data
Aug. 23, 2004 (RU) 2004125522

(51) Int. Cl. C12Q 1/02

(52) U.S. Cl. 435/29

(57) ABSTRACT

Erythrocyte sedimentation rate (“ESR”) determinations of a patient's blood that has been combined either with blood serum from pregnant mammals or with fetal embryonic serum yield measurable results correlating well with the presence of an on-going cancer. This cancer detection method is based on differential ESR determinations and uses paired in vitro ESR tests, in which a patient's whole blood is combined with a control serum (in a control vial) comprising serum from non-pregnant mammal and with a test serum (in a test vial) comprising either fetal embryonic serum or serum from a pregnant mammal to measure differential ESR in the tested control vials. Cancer coefficient K is calculated based on the ESR measurements and is compared with a threshold value to determine whether the patient may be identified as possibly having an on-going malignancy. A kit for performing the cancer screening methodology is also provided.

### REFERENCE TO RELATED APPLICATION

This patent application is a Continuation-In-Part of Ser. No. 11/138,334 filed on 27 May 2005.

### FIELD OF THE INVENTION

The present invention is directed to medical and veterinary diagnostic tests, such as immunologic tests for the presence of neoplastic diseases in humans and other mammals, and particularly, to cancer screening laboratory tests permitting cancer presence determination and measurements of cancer aggressiveness.

The present invention is further directed to a cost-effective oncology screening protocol comprising an in-vitro methodology using erythrocyte sedimentation rate determinations, as well as fetal embryonic serum and serum from pregnant mammals to screen test subjects for on-going neoplastic processes.

In overall concept, the present invention is directed to an in vitro medical cancer screening test that compares erythrocyte sedimentation rates (ESR) in blood of a test subject mixed with an aliquot of embryonic fetal serum, or with an aliquot of serum from a pregnant mammal, with a control erythrocyte sedimentation rate to establish the probability of cancer presence in a patient.

### BACKGROUND OF THE INVENTION

Presently, many cases of cancer remain undetected until severe symptoms are manifested. At the later stages of cancer too many cases are refractory to present treatments. Therefore, early diagnosis of cancer is extremely important, since the outcome of cancer treatment depends on the stage of cancer when detected. Accordingly, a reliable test for early cancer detection would help enormously since the survival rate with modern cancer therapies in cases of early detection is higher than 90%.

There exist numerous tests to screen patients for cancer. Most of them are concerned with screening for a particular type of cancer. In vitro tests are used to indicate the presence of a malignant neoplastic process within a patient but these all have in common problems of poor specificity, and/or sensitivity, and/or predictive value.

Since its introduction in the early 1920's, the erythrocyte sedimentation rate (“ESR”) has been used in various ways for screening of patients with suspected malignancies. However, the lack of predictability and poor specificity of the ESR tests, as well as excessive sensitivity to both malignant and non-malignant pathologies prevent reliable and straightforward use of the ESR for cancer screening. On average, the current ESR tests miss 25% of those with established neoplastic malignant disease diagnoses and give a ‘false negative’ result. On the other hand, the ESR tests may also give an elevated reading in numerous non-cancer inflammatory conditions, also known as ‘false positives’.

The ESR test is a laboratory test using the whole blood of a patient or mammal. The collected blood is anti-coagulated and then introduced into standardized capillary tubes for ESR determination. The ESR measures the volume of plasma above the column of settled blood cells after the red blood cells have been allowed to settle for a predetermined amount of time, typically an hour or less in a properly calibrated tube. Certain technical factors in the ESR test affect the aggregation of red blood cells to rouleaux or stacked formations, including the particular characteristics of the erythrocytes such as size, shape and surface charge, the viscosity of the plasma, and the interacting electrostatic forces of the surrounding macromolecules, notably fibrinogen, albumen and gamma globulins.

Erythrocytes normally repel each other as a result of the negative charges of the carboxyl group of N-acetylneuraminic acid located on the surface of the red blood cell. Aggregation of erythrocytes is increased when these negative charges are neutralized to a greater or lesser extent by circulating plasma macromolecules, thereby promoting rouleaux formation, as well as adding weight to the red blood cell by the adhesion of these circulating plasma macromolecules. The result of the process is an increased rate at which the erythrocytes fall to the bottom of the capillary tube used in ESR determination techniques, hence an increased ESR value.

ESR determinations are quite sensitive to extrinsic factors, such as room temperature, the amount of anticoagulant added to the specimen to prevent clot formation, the length of time between sample collection and testing, the deviation of the test tubes from a strictly upright position, the presence of any bubbles in the test tube, and the length of time at which the ESR value is established. These extrinsic factors may be controlled to a great extent by consistent procedural standards.

ESR determinations may be abnormally elevated as an indication of a diverse range of on-going diseases, which has led clinicians in the past to conclude that the ESR determination test was, at best, a marginally useful diagnostic test. Nonetheless, the simplicity and low cost of ESR determination testing have prompted numerous investigators to try to adapt the ESR for diagnostic applications in ways that improved sensitivity, specificity and predictive value of the ESR technique.

The ESR techniques use test serum. Prior art methods of producing suitable test serum are extremely complicated as they require the double immunization of small syngeneic laboratory mammalian animals. Furthermore, the use of small laboratory mammals does not support the commercially viable production of substantial amounts of fetal embryonic test serum. Additionally, these same prior art methods are extremely difficult to apply to large mammals.

Very often the virulence, the relative malignancy of a particular tumor is not very evident. For example, about one out of ten cases of breast cancer is particularly dangerous, referred to often as a high grade malignancy associated with an anaplastic histology and poor prognosis, and there is no current technology on the market to differentiate which cases of breast cancer are especially dangerous and which are not. It is common practice for doctors to recommend mastectomy if any doubt exists. If a cancer test existed which could give information as to what level (or grade) of the malignancy is present, many patients might be spared a mastectomy, which might not be indicated in a substantial number of cases.

Measurement of the aggressiveness of a detected cancer is of critical importance as it gives physicians the ability to more fully appreciate the type of cancer requiring treatment. Moreover, it would allow physicians to forecast the effectiveness of a particular treatment by monitoring the cancer coefficient. For example, if a treatment is effective, it is to be expected that the cancer coefficient will decrease. If a particular treatment is ineffective, the converse would be true and a different course of treatment could be evaluated and implemented. A need exists for a test to measure the aggressiveness of a detected cancer and no such test is currently known in the art.

The timely monitoring of the effectiveness of cancer treatments is very important. For example, the size of a tumor and extent of a cancer's aggressiveness may not be completely determinable prior to a surgical effort to remove all of the tumor. Complete removal of a tumor can revert ESR-based cancer coefficient to a negative result within about two weeks of complete tumor removal. Whether the screening test remains positive or reverts to negative may be an important factor in decisions regarding post-operative radiation and/or chemotherapy. A reliable timely cancer test is needed which could serve for such applications.

New medications for treating cancers are continuously being developed and it takes enormous expenditures to determine the true effectiveness of these new medications. Moreover, the numerous new products and medicines being developed have to undergo additional testing for any carcinogenic potential, which is a long and costly process.

Therefore, an effective cancer screening technique which would permit to determine the presence or absence of disease, to monitor the progression or improvement of an already diagnosed disease, to measure the response to treatments, to shorten and economize carcinogenicity screening trials for drugs and chemical products, as well as to permit a reliable cancer determination at early stages and its aggressiveness, is a long-lasting need in diagnostic medicine.

### SUMMARY OF THE INVENTION

It is therefore an object of the present invention to provide meaningful and reliable cost-effective diagnostic technique to determine the presence or absence of a neoplastic disease process in a patient, as well as to measure the aggressiveness of the detected cancer, and capable of monitoring the response to treatments in a most effective, rapid and inexpensive fashion.

It is a further object of the present invention to provide malignant tumor screening in which a commercially available source of embryonic fetal serum or its equivalent is used, such as serum from pregnant mammals. The test serum, in this case the embryonic serum, can be produced in sufficiently large quantities and thereby transform a cumbersome and labor intensive laboratory effort into a practical clinical screening test.

The subject invention's method of measuring cancer aggressiveness and detection thereof for humans and other mammals preferably comprises the steps of:
combining a predetermined amount of an fetal embryonic or pregnant animal serum with a sample of a patient's whole blood,
combining, for the test's control, blood serum from a healthy non-pregnant mammal with another aliquot of the patient's whole blood;
establishing ESR value for each of the two blood samples tested; and
calculating a cancer coefficient based on the measured ESR values.

The difference between the two ESR determinations results allows the patient-specific determination of a cancer coefficient, along with the concomitant probabilities of an on-going malignant neoplastic process. The likelihood of an on-going neoplastic pathology is a probability value between zero and one assigned by using the cancer coefficient and nomograms derived from historical data matching cancer status with measured cancer coefficient.

Serum that is obtained from the blood of pregnant mammals may be taken anytime during pregnancy but preferably during the second trimester, and more preferably between around the 45^{th} and 100^{th} day of gestation. The pregnant horse serum is then combined with an aliquot of a patient's blood, and an ESR determination is made. Then the differential ESR determination test of the present invention allows the quantification of the probability of an on-going cancer according to the difference in the ESR determinations between testing and control results.

Blood serum from various pregnant mammals, including horses, cows, sheep, goats, etc., can be used as the test material. Alternatively, embryonic fetal serum can also be used as the test material for the screening test. The use of different pregnant mammals' blood serum and of different embryonic serum provides flexibility to the inventive screening test. It has been found that the more extensive testing, which includes the separate use of different sera at the same time, further improves the sensitivity and predictability of the test methodology in question.

The present screening technique does not rely upon the information regarding the specific tumor histology or anatomic location, and therefore is not sensitive to a malignant neoplasm's anatomic location or its particular histology.

### BRIEF DESCRIPTION OF THE DRAWINGS

FIG. 1 illustrates the statistical coefficients of malignancy for various cancers determined by the method of the present invention; and

FIGS. 2A-2D illustrate schematically the sequence of steps of the method of the present invention.

### DETAILED DESCRIPTION OF THE PREFERRED EMBODIMENT

In general, the subject cancer screening methodology is based on the ESR platform and exploits the separation between erythrocytes and plasma over time. It is well known, that in addition to other functions, erythrocytes act as messengers in the body by carrying on their surfaces antigens from different cells encountered in the body. Antigens on the surfaces of erythrocytes may include the tumorous antigens from cancer cells. These tumorous antigens, which are early embryonic antigens, are generated during cancer development. It appears that early embryonic antigens are highly conserved among all animal species.

It is the Applicant's belief that the theoretical basis for subject cancer screening test suitable for mass production is the parthenogenesis hypothesis of oncogenesis. According to this hypothesis, malignant changes happening in a differentiating somatic cells cause activation of a cellular genetic mechanism, via parthenogenesis, which switches on two successive genetic programs: (1) the program for normal development of a somatic cell blocked at some stage of differentiation and (2) the program for normal oncogenesis which starts from the very beginning. The last program for normal oncogenesis starts only for some cells.

This theory is supported by a number of experimental facts. For example, it has been found that malignant tumors consist of two different kinds of cells: (1) one type of cells are similar to cells of normal tissue from which the tumor originated (tumor stem cells), and (2) the second type of cells (differentiating from tumor stem cells) are similar to normal cells from which the tumor originated but which with time, become more and more similar to the differentiated cells of other organs and tissue. Rates of such progression will be different and highest for poorly differentiated (anaplastic) tumors, medium for moderately differentiated tumors, and low for highly differentiated tumors. It is believed that all carcinogenic substances, including chemical agents and viruses, trigger this paraparthenogenetic mechanism, which is the same for all tumors. For reasons unknown, oncogenesis does not start in non-malignant tumors.

The following evidence favors the parthenogenesis hypothesis:
1) It was shown experimentally that Teratocarcinoma of the testicle in both humans and animals consists of a mixture of cell types: one cell type is similar to testicular cells of an embryo, and the other cell type has similarities to 14 types of cells of different organs and tissues. It was demonstrated that if one cell of the first type is implanted it triggers growth of the same teratocarcinoma, and if other type cell is implanted it does not trigger tumor growth;
2) Expression of oncogenes (c-srk, c-myc, c-erb, crash, c-resk, c-sis, N-myc, L-myc) has been demonstrated with tumor growth and with the early stages of embryogenesis. It seems that oncogenes are normal components of embryogenesis;
3) Messenger RNA (mRNA) found in a cancer cell cytoplasm is very similar to embryonic mRNA at early stages of growth;
4) Cancer cells generate products which typically are found in embryonic cells during early stages of growth. For a human embryo this period starts at gametogenesis and continues up to 10 weeks of growth. When the genesis of organs is finished, generation of at least some products common for embryo cells and for cancer cells also stops.

A central tenet of the parthenogenesis hypothesis is that cancer cells even at early stages of tumor growth produce early embryonic antigens (antigens specific for a stage of growth). A basic bio-genetic law states that during development of each individual, a history of genesis of all ancestors of this individual repeats in short and compressed issue, e.g., early embryonic antigens and their associated antibodies have been highly conserved throughout evolution and remain so in all animal species. For example, it was demonstrated that during human embryogenesis, the progression of time, such antigens appear as are typical for frog, snake, and only later for humans. In other words, early embryonic antigens and therefore antibodies to them are similar in all animals and this is one of the basic concepts of the invented test.

Many similarities have been found between malignant tumor cells and embryonic cells during the early stages of their development. For example, it is known that blood serum from rabbits immunized with rat embryo cells react positively in binding with lipoid extract from rat placental tissue, lipoid extract from Jensen sarcomas, and an even more positive binding reaction with lipoid extract from human cancer tissue. At the same time, this serum did not react with extracts from normal tissue. This result has been confirmed.

It is believed that embryonic blood antigens, including antigens apparent during the early stages of embryogenesis (early embryonic antigens, stage specific antigens) and which do not exist in mother blood, can pass the placenta and cause the generation of specific antibodies in mother blood. Such specific antibodies are found in all cases of normally progressing pregnancy, and according to some author's opinion, they positively regulate and normalize growth of an embryo. There are cases, however, when during a seemingly normal pregnancy, an “immunologic conflict” occurs between a mother and her embryo. It happens sometimes with pregnant horses. Such cases occur particularly often if a mare is coupled with an ass and the embryo acquired antigens from the ass. These mare immunizing antigens generate specific antibodies, which may kill an embryo.

It is believed that antibody generation starts a chain of immunologic reactions. For example, the immune response to antigen are antibodies that may have autoimmunogenic idiotypical determinants. In other words, each antibody (At1) may induce the generation of anti-idiotypical antibodies (At2), which in their turn induce the production of anti-anti-idiotypical antibodies (At3), which in their turn may induce the generation of anti-anti-anti-idiotypical antibodies (At4), etc.

Anti-idiotypical antibodies (At2) and anti-anti-anti-idiotypical antibodies (At4) carry an “antigen internal design”, i.e. they have antigen determinants that can bind with At1 and At3. It has been shown that antigens and idiotypes are expressed on antigen binding receptors of T, erythrocytes and B-lymphocytes, and that they are expressed on the outer membranes of erythrocytes, and further that there are antigens and idiotypes in blood serum.

The facts presented in the previous paragraphs support the consideration that in a cancer patient, early embryonic antigens (stage specific) are generated, which induce the generation antibodies (At1) to early embryonic antigens, which in their turn cause the generation of anti-idiotypical antibodies (At2), which in their turn cause the generation of anti-anti-idiotypical antibodies (At3), etc. These early embryonic antigens and At1, At2, At3, At4, etc., are expressed on T and B-lymphocytes, on erythrocytes, and are present in blood serum of cancer patients. Thus, the antigens and anti-idiotypes of anti-embryonic antibodies present in the test serums reacts with embryonic-type antigens found in a cancer patient's whole blood and affects the ESR reaction in a quantifiable manner.

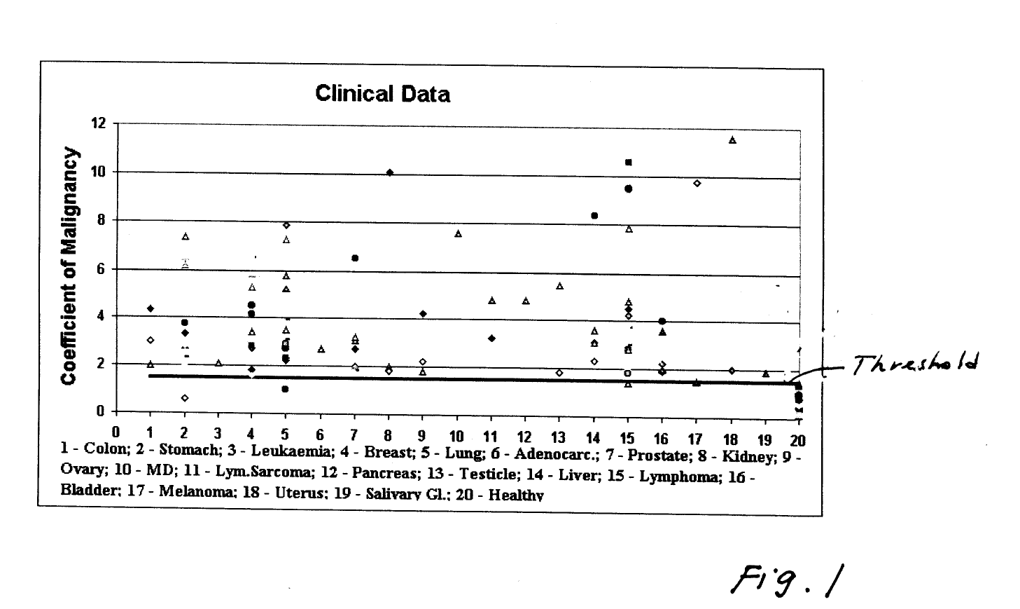

The terminology of “patient” used in this method for the detection and aggressiveness of cancer shall be understood to be a human being or any other animal. Additionally, venous arterial or capillary whole blood may be used for the cancer aggressiveness and detection system and method. In overall concept, a cancer coefficient value (K) is determined by the use of the method and system as is shown in the following paragraphs. It has been found that a cancer coefficient (K) above a predetermined threshold (as shown in FIG. 1) indicates that cancer may present in the patient. Further, the value of the cancer coefficient (K), with an absolute value higher above the predetermined threshold of 1.5, is an indication not only of presence/activity of the cancer in the patient, but also of a higher aggressiveness of the cancer.

The test of the present invention includes the steps of: acquiring a sample of blood from a patient and combining that blood with the serum from either a pregnant animal or an animal embryo. A second blood sample from the patient is then combined with serum of a non-pregnant animal of the same species.

At this point, there are two separate and distinct blood mixtures. In one mixture, patient erythrocytes with different antigens expressed on antigen binding receptors of erythrocytes, including the early embryonic antigens (if there is a cancer), are combined with antibodies to the early embryonic antigen via serum of either the pregnant animal or animal embryos. In another mixture, the erythrocytes with different antigens expressed on antigen binding receptors of erythrocytes, including the early embryonic antigens (if there is a cancer), are combined with serum with no antibodies to the early embryonic antigen; thus no antibodies to the early embryonic antigen exist in the second mixture.

The tumorous antigens, which are early embryonic antigens, react with the early embryonic antibodies. Accordingly, if the early embryonic antigens exist on the surface of a patient's erythrocytes (there is a cancer), then a reaction will occur between the early embryonic antibodies added to a patient's blood and the patient's erythrocytes. Specifically, that reaction building bridges between erythrocytes cause the patient's erythrocytes to adhere to one another, and that in turn, causes the erythrocytes to become sedimentary at a rate much faster than the erythrocytes in the second mixture where no early embryonic antibodies are present for reaction with erythrocytes. Erythrocytes having tumorous antigens on their surface more actively separate themselves from plasma in the presence of early embryonic antibodies. This can be empirically and visually observed by comparing the difference between the two ESR reactions.

Results of this test can be extrapolated and interpreted in the following fashion: as the number of erythrocytes having tumorous antigens on their surface increases, so will do the difference between the ESR reactions. Since generation of antigens depends on biological activity of cells, it may be logically assumed that the density of tumorous antigens is directly proportional to the aggressiveness or malignancy of detected cancers, and it is through this test, that we are able to measure the malignancy of detected cancers.

As demonstrated in FIG. 1, the experimental data presents different results for the same type of cancer. For example, for N=1 at the horizontal axis of the diagram, which corresponds to a colon cancer, there are three different cancer coefficients (on the vertical axis of the diagram) for three different cases of the same colon cancer, e.g. 2, 3 and 4.3. These results demonstrate that the three cases of colon cancer are proceeding in different ways, namely, the colon cancer with a cancer coefficient of 4.3 is more aggressive than in the other two cases where the cancer coefficient is 2 and 3 respectively.

Also, it is to be noted that a threshold is found when resulting coefficient of malignancy K is equal to 1.5 as shown by the threshold line in FIG. 1. The experimental data supports an assumption that this number is a threshold number for the present test and if this resulting coefficient K is bigger than 1.5, it may be said that cancer is detected. If the coefficient present test has higher value, it may be interpreted that malignancy of the detected cancer is higher.

The utility of a diagnostic test is characterized by the sensitivity, specificity, and positive predictive value of the test. The quality and utility of the subject cancer screening methodology when used in one series of clinical investigations is summarized in Table 1, which relates to the clinical data presented in Table 2.

In the test performed, the number of patients who are test positive and who also have a neoplastic disease is designated ‘A’; those who are test positive but who do not have cancer are designated ‘B’; those who have a neoplastic disease but whose test results are negative are represented as ‘C’; and those with a negative test result who do not have a neoplastic disease are designated ‘D’.

TABLE 1

| TEST | DISEASE | DISEASE NOT |
|---|---|---|
| RESULT | PRESENT | PRESENT |
| Positive | A (n = 915) | B (n = 0) |
| Negative | C (n = 96) | D (n = 24) |

The sensitivity of a diagnostic test is defined as the probability that when the disease is present the test results will be positive; sensitivity of the test is calculated as A divided by the sum A+C [Sensitivity=A/(A+C)].

The specificity of the test is defined as the probability that the test is negative when no disease is present; the specificity is thus calculated by dividing B by the sum B+D [Specificity=D/(B+D)].

The positive predictive value of a test is the probability that a positive test result reflects the presence of disease [Positive Predictive Value=A/(A+B)].

The data presented in Table 1 indicate for the differential-ESR diagnostic test values while practicing the preferred methodology of the present invention a test Sensitivity of 0.9, a test Specificity of 1.0, and a Predictive Value of 1.0, indicative of a significant degree of clinical usefulness for the subject inventive laboratory medical test methodology.

TABLE 2

| Cancer Diagnosis | # of Patients | Test Positive | Test Negative |
|---|---|---|---|
| Breast | 207 | 186 | 21 |
| Lung | 478 | 429 | 49 |
| Gastric | 95 | 87 | 8 |
| Enteric | 36 | 31 | 5 |
| Colon | 49 | 45 | 4 |
| Renal | 23 | 21 | 2 |
| Bladder | 41 | 38 | 3 |
| Gonadal | 22 | 20 | 2 |
| Thyroid | 15 | 14 | 1 |
| Leukemia | 1 | 1 | 0 |
| Prostate | 9 | 9 | 0 |
| Adenocarcinoma | 1 | 1 | 0 |
| Multiple Myeloma | 1 | 1 | 0 |
| Lymphosarcoma | 4 | 4 | 0 |
| Pancreatic | 1 | 1 | 0 |
| Hepatic | 5 | 5 | 0 |
| Lymphoma | 17 | 16 | 1 |
| Melanoma | 2 | 2 | 0 |
| Uterine | 3 | 3 | 0 |
| Salivary Glands | 1 | 1 | 0 |
| Chapter 2 No | 24 | 0 | 24 |
| Cancer (Healthy) | | | |
| Total | 1011 | 915 | 96 |

An important concept of the subject invention is that blood serum taken from pregnant mammals or embryos and combined with whole blood of a patient can yield a measurable result, e.g., a cancer coefficient, by using the ESR methodology combined with an immunologic reaction. The differential ESR technique compares the ESR of a patient's whole blood when combined with blood serum of a pregnant mammal or fetal embryonic serum, with the ESR of a patient's blood when combined with control serum from a non-pregnant mammal. The same inventive concept applies to tissue suspensions and other bodily fluids, but the immunologic reactions associated with an on-going neoplastic disease must be detected by other methods, such as immunofluorescence.

In order to improve the accuracy of resulting determinations, an extended differential type of reaction is employed. For example, two separate samples of patient blood are obtained and then two types of mammal serum are added to the two acquired patient samples: (1) test serum obtained from a pregnant mammal is combined with a patient's first blood sample, and (2) control blood serum taken from a non-pregnant mammal of the same species is added to a second blood sample of the same patient.

If the difference in reactions in two or more samples, the extended differential ESR value that is directly reflected in the cancer coefficient, reaches a predetermined threshold value for the cancer coefficient, typically greater than 1.5 in the preferred embodiment, then the subject screening methodology is positive for an on-going malignancy. The cancer coefficient value is directly proportional to the ESR difference in millimeters mathematically weighted (by multiplication) with the maximal ESR measure and normalized to discount the various particular capillary tube dimensions.

The inventive methodology uses a pregnant mammal's blood serum as a test serum in contrast with a control serum to determine vel non a presumptive cancer diagnosis. For example, when blood serum of pregnant mares is used as a test serum, then blood serum from a normal non-pregnant horse is used as a control serum.

In the subject method, the erythrocyte sedimentation rate (ESR) is defined as a sedimentation rate, e.g. the rate at which red blood cells precipitate in a predetermined time interval, commonly one hour. In order to obtain test results, anticoagulated blood is placed in an upright (or tilted) tube, and the rate at which the red blood cells fall is measured, is defined as the erythrocyte sedimentation rate (ESR), in a linear dimension per time, normally millimeters per hour (mm/h).

The subject invention system and method utilizes two ESR rates. The first ESR rate (ESR₁) is developed by mixing a patient's anticoagulated whole blood with the serum of a pregnant animal (test serum). In the preferred embodiment, the test serum is obtained from a pregnant mare preferably during the second trimester of pregnancy and more preferably in the 45^{th} to 100^{th} day of gestation.

The second erythrocyte sedimentation rate (ESR₂) is developed by mixing the patient's anticoagulated whole blood with the serum of a non-pregnant animal species (the normal/control serum) that is the same species of animal used to develop the first ESR rate (ESR₁). In a preferred embodiment, a non-pregnant female animal is used, however, it is envisioned that a male animal could also be used.

Importantly, if the blood serum used for the test serum of the pregnant mammal is from an animal, including but not limited to, a pregnant cow, dog, sheep, goat, pig, etc., then a serum from a non-pregnant cow, dog, sheep, goat, pig, etc, respectively, must be used as the control serum. In the preferred embodiment, however, fetal embryonic blood serum is used as the test serum and normal serum of the same mammal species used as the control serum. In other embodiments, blood serum of a horse, cow, dog, sheep, goat, etc. or embryo is used as the test serum and the normal blood serum of the horse, cow, dog, sheep, goat, etc. is used as the control serum. The pregnant animal serum may be substituted for with fetal embryonic serum in each of the cuvettes containing patient's blood in the ESR₁ and ESR₂.

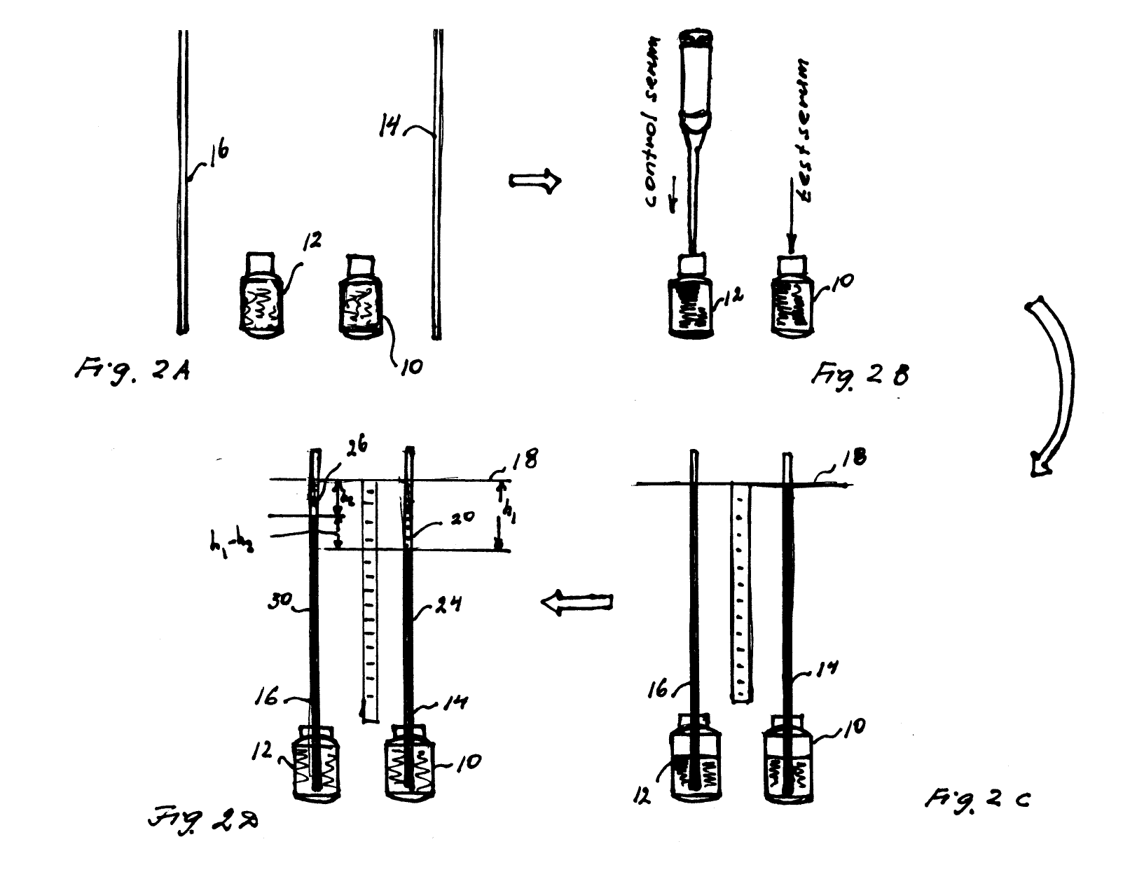

The method for determining the existence of cancer and/or the aggressiveness of cancer in a patient includes the preparation of a predetermined amount of heparinized blood, for example, 200 microliters (“μL”) of heparinized blood for glass capillary tube with the internal diameter of 1.2 mm, and, for example, 400 microliters (“μL”) of heparinized blood for Sediplast® ESR system capillary tube with the internal diameter of 2.55 mm, by adding a predetermined amount of lithium heparin for every 1 ml of blood. For example, with the glass capillary tube (internal diameter of 1.2 mm) approximately 20 units of lithium heparin may be used. The heparnized blood is then placed into each of two ESR cuvettes 10 and 12, as shown in FIG. 2A.

As shown in FIG. 2B, a predetermined amount of test serum (approximately 50 μL if a glass capillary tube having internal diameter of 1.2 mm is used; and 100 μL if the Sediplast® ESR system capillary tube having internal diameter of 2.55 mm is used) is added to the cuvette 10 containing the patient's blood sample and a predetermined amount of a control serum is added to the cuvette 12 containing the patient's blood sample (approximately 50 μL for a glass capillary tube with the internal diameter of 1.2 mm, and 100 μL for the Sediplast®t ESR system capillary tube with the internal diameter of 2.55 mm).

The cuvettes may then be shaken for a predetermined time, for example, approximately 10 seconds, which is done typically by inverting the tube four or five times or by using a mechanical shaker.

For the same test, it is preferred that two identical capillary tubes 14, 16 are inserted into the cuvettes 10, 12 containing the test blood-serum mixture and the control blood-serum mixture respectively. When inserted into the cuvettes, the capillary tubes become filled with the respective blood mixture contained in the cuvettes 10, 12 until substantially the same surface level (“zero line”) 18 in both capillary tubes has been reached, as shown in FIG. 2C. The capillary tubes may be positioned vertically or, in an alternative preferred embodiment, tilted at a pre-determined angle away from the vertical position, as will be described in the following paragraphs.

Although capillary tubes may be used from many different manufactures, as an example, the standardized Sediplast® ESR system capillary tubes were used in conducting the present method. A 50 mm long glass capillary tube with an internal diameter of 1.2 mm may be used and maintained preferably at body temperature if approximately 37° C. Lower temperatures including room temperatures ranging between 20-22° C. may be used, however, with a somewhat longer reaction time.

After the stage of the subject method shown in FIG. 2C, an elapse time period is applied to the test system, as presented in FIG. 2D. During the elapse time period, the test blood-serum mixture in the test tube 14 is divided into the clear plasma column 20 having the height h₁ and the sedimented erythrocytes column 24, while in the control tube 16, the control blood-serum mixture is separated into the clear plasma column 26 having the height h₂ and the sedimented erythrocytes column 30.

It is important that the elapse period is of such a duration, that the measurements of h₁ and h₂ are taken for the calculation of the cancer coefficient when |h₁−h₂| is at its maximum, e.g., when the ESR reaches a “plateau” in the erythrocytes rouleaux formation. At this ESR “plateau”, the h₁ and h₂ do not change, and |h₁−h₂| is at the maximum. The elapse period prior to reaching the maximum difference between h₁ and h₂, may change dramatically between the patients. For example, for human patients, the elapse time period may range between 30 min and 1 h, or may even reach 1-2 hours. For dog testing, the elapse time period may range between 1.5 hours and 5 hours.

In order to precisely determine the elapse time period before measurements of h₁ and h₂ are to be taken, e.g., to assure that h₁ and h₂ are measured at the ESR “plateau,” approximately 15 min after the mixtures in the capillary tubes 14, 16 have reached the “zero level” 18, the height (h₁) of the clear plasma column 20 in the capillary tube 14 and the height (h₂) of the clear plasma above the sedimented erythrocytes 30 in the capillary tube 16 are measured periodically, for example, every 3-5 minutes until maximum difference in the heights h₁ and h₂ (h₁−h₂) in the capillary tubes 14, 16 is attained. The measurements of h₁ and h₂ taken when their difference is at its maximum, are used for determination of the cancer coefficient.

ESR values (ESR₁ and ESR₂) may be determined by measuring h₁ and h₂ in millimeters which correspond respectively to ESR₁ and ESR₂ of erythrocyte sediment per hour.

In order to increase the accuracy of the test, more than one pair of capillary tubes may be used for the same patient. When using more than one pair of capillary tubes, the arithmetic average of measurement results within each test group may be used.

Using the values h₁ and h₂, a presumptive cancer diagnosis may be made according to the formula for a differential ESR derived cancer coefficient, as presented in further paragraphs.

The difference in the ESR₁ and ESR₂ is established in FIG. 2D as the difference between h₁ and h₂, and a cancer coefficient K may be calculated according to an empirically derived formula (Eq. 1) of the subject invention.

K=[(ESR₁−ESR₂)×ESR_{max]/NF} (Eq. 1)

where:
ESR₁=the erythrocyte sedimentation rate of the mixture of the patient's anticoagulated whole blood and pregnant animal serum,
ESR₂=the erythrocyte sedimentation rate of the mixture of the patient's anticoagulated whole blood and non-pregnant animal serum,
ESRₘₐₓ=the greater absolute value of ESR₁ and ESR₂, and
NF is a normalization factor described in detail in further paragraphs.

The Eq. 1 may be re-written to accommodate the measurement data of the test:

K=[(h₁−h₂)×h_{max]/NF} (Eq. 2)

wherein:
h₁ is the measured height of the column of the clear plasma in one capillary tube,
h₂ is the measured height of the column of the clear plasma in another capillary tube, and
hₘₐₓ is the greater of h₁ and h₂.

The normalization factor (NF) depends on the internal diameter of the capillary tube and has been empirically derived as:

NF=A×(D)² (Eq. 3)

where: A=35.4, and
D=internal diameter of capillary tube (mm).

Different capillary tubes having differing internal diameters may be used, however, it is of importance that a pair of identical capillary tubes with the same internal diameters are used for the same test.

To find the quantitative dependence of the normalization factor NF from the internal diameter of the capillary tube used in the test, the Applicants have collected and analyzed a tremendous amount of test data for patients free of cancer for a variety of capillary tubes of different internal diameters. As the result, it has been found that for example, when a 1.2 mm glass capillary tube is used, the normalization factor is approximately 50. When a Sediplast® ESR capillary tube system is used having a 2.55 mm internal diameter, the normalization factor has been found to be 230. From these findings, the coefficient A has been derived

$$A = \frac{N F}{{D}^{2}}$$

by calculations to be A=35.4.

Once the normalization factor NF has been found for a specific capillary tube's diameter (Eq. 3), the cancer coefficient (K) can be derived from the (Eq. 2).

In overall concept, the subject inventive methodology encompasses substantially equivalent cancer coefficient expressions using formula that mathematically weigh the difference between the test ESR (ESR₁) and the control ESR (ESR₂) determinations by multiplying such by a maximal ESR (the maximum absolute value between the maximum of ESR₁ and ESR₂) value in the test group and normalizing the expression for the capillary tube dimensions (internal diameter). Calculated Cancer coefficients were compared statistically with historical clinical data of cancer coefficients from patients with established cancers to provide a probability of the presence of cancer and/or the aggressiveness of a particular cancer in a particular patient. Returning to FIG. 1, it is clearly presented that patients having more aggressive cancers have the cancer coefficients greater than 1.5, which is the threshold value for diagnosing cancer under the present methodology. Accordingly, a relationship between the cancer coefficient and the aggressiveness of a diagnosed cancer exists wherein the greater the cancer coefficient, the more aggressive the cancer. The cancer coefficient value greater than 1.5 is associated with a substantial probability (about 85%) that there is an ongoing malignancy in the patient. In general, higher cancer coefficients correlate with higher probabilities of ongoing, more aggressive, and higher grade malignancies. Cancer patients having cancer coefficients reaching 4.0-5.0 and higher have been biopsy diagnosed. Cancer coefficient values greater than or equal to 4.0 are associated with a much higher probability of on-going malignancy.

In order to improve the sensitivity and specificity of the test, preferred embodiments of the subject inventive cancer aggressiveness and screening methodology include the use of blood serum from pregnant mammals of different species and embryonic blood serum, as the test serum, separately for the same patient evaluation, with non-pregnant serum of the same mammalian species as the control serum. For example, the blood serum of a pregnant horse and the blood serum of a pregnant cow can be used separately as test sera. Sera from different mammals may also be used additionally during the screening test to further optimize its specificity, efficiency and reliability.

Different anticoagulants may be combined with whole blood, either manually or by using commercially available collection tubes which contain an anticoagulant. The exemplary tests discussed below all were performed using a “Green Top” collection tube with a 4.5 ml capacity and containing Lithium Heparin (72 units) as an inside coating. Lithium Heparin was used at about 13 to 20 units per ml of whole blood.

Alternatively, Sodium Heparin may be combined with whole blood with a stoichiometry in the range of about 12 to 20 units per ml of blood. Other anticoagulant used for this method step may also include Ammonium Heparin.

It is contemplated within the scope of the present subject invention that alternative approaches for the ESR determination may be used. For example, anticoagulants providing functional equivalence may be substituted for the preferred Lithium Heparin without any noticeable effect on the present cancer screening protocol. Also, the test serum and the control serum may be loaded into blood collection tubes during their manufacture.

The present subject invention may encompass the use of tilted capillary tubes during the erythrocyte sedimentation phases in the ESR methodology. Specifically, the capillary tubes may be tilted an angle between about 10° and about 55°, preferably at 45°. It has been observed regarding the vertical position thereof that the rate of ESR equilibration, that is the time until the ESR test reaches a plateau in erythrocytes rouleaux formation and settling, is roughly halved when the capillary tubes are tilted at a 45° angle.

Without intending in any way to be bound by theory, it is believed that the adhesion of gamma globulins to erythrocytes occurs primarily at surface layers where the force of surface tension accelerates the molecular interactions. When a capillary tube is tilted to a 45° angle, the surface area inside of the capillary tube doubles. This correlates well with the inventors' observation that ESR determination tests can be done in roughly half the time when a capillary tube is tilted to a 45° angle compared with that of the customary upright position without any degradation of specificity, sensitivity, predictability or reliability.

The subject invention in addition to be a method for determining the existence of cancer in a patient and/or the aggressiveness of the detected cancer, also encompasses a diagnostic screening kit for performing the cancer screening methodology disclosed and claimed. The kit may provide at least two kinds of serum: a first fluid designated ‘A’ referred to as the test serum, and a second fluid designated ‘C’ referred to as the neutral or control serum, which is to be combined with a patient's heparinized specimen. The sera can be provided for the test in a liquid, frozen or freeze-dried form and can further be provided in some commercially available vials.

The kit further may comprise:
1) At least one test tube when using SEDIPLAST® (Polymedco, Cortland, N.Y.) plastic 100 mm long capillary tubes or at least two test tubes when using glass 50 mm long glass capillary tubes, preferably pre-loaded during manufacture with a predetermined effective amount of anticoagulant for the patient's blood specimen;
2) At least two standardized capillary tubes preferably the commercially available SEDIPLAST® ESR System plastic capillary tubes; other types of capillary tubes can be used, such as glass, but different volumes of blood will be needed.
3) At least two vials, equivalently referred to as cuvettes, for blood mixing, as are supplied with “SEDIPLAST®” capillary tubes;
* optionally included equipment may comprise:
4) Laboratory pipettes with 40 to 500 μL capability;
5) Test tube stands or other support for the capillary tubes.

The preferred embodiment of the subject invention contemplates that the methodology will be practiced in a facility equipped with a vibrator-mixer for blood serum mixing, and a thermostat-regulated ambient temperature about 37° C. that allows shorter test times.

### DESCRIPTION OF ILLUSTRATIVE EMBODIMENTS

83 clinical examples of the use of the present inventive cancer screening presented in the following paragraphs demonstrate the efficacy of the subject methodology in detection of a wide variety of on-going cancers. These illustrative embodiments further emphasize the usefulness of the present subject cancer screening test for humans and in veterinary applications.

The following are clinical examples of the cancer screening using the subject inventive methodology for sixty human and twenty-three canine patients for an on-going malignancy. Each of the sixty clinical examples below was carried out by practicing the same method steps, namely:
Aliquots of about 3-4 ml of whole blood were obtained by venipuncture from each patient and transferred to a 4.5 ml “Green Top” blood collection tube containing 72 units of Lithium Heparin as the anticoagulant. Brief shaking for a few seconds assures adequate mixing of the blood and anticoagulant.
About 400 μL of patient blood was combined with about 100 μL of pregnant horse serum in cuvette which was gently shaken by hand for about half a minute and then transferred to SEDIPLAST® (Polymedco, Cortland, N.Y.) plastic 100 mm long capillary tube and left standing at room temperature of about 21° C. in the vertical condition for about 45 minutes; and
About 400 μL of patient blood was combined with about 100 μL of non-pregnant horse serum in a second SEDIPLAST® plastic 100 mm long capillary tube, shaken by hand for about half a minute and left standing at about 21° C. in the vertical condition for about 45 minutes.
In each case, about 400 μL of patient blood was combined with about 100 μL of serum. Measurements were taken of the height (h₁, h₂) of the serum—erythrocyte sediment interface from the top of the meniscus (line 18 in FIG. 2C) in the capillary tube and recorded as the particular ESR value (ESR₁, ESR₂) for that part of the cancer screening to be used in the cancer coefficient calculations.
The maximal ESR value E_{Max} corresponding to the maximal of two measured h₁ and h₂ for the group of ESR determinations were established for each patient.
Cancer coefficients for each patient's were established according to the formula:

K=[(h₁−h₂)*hₘₐₓ]/NF where the normalization factor NF=230 mm².

It is to be noted that the height of blood mixture in capillaries should be about 50 mm for the glass capillary and about 100 mm for SEDIPLAST® plastic 100 mm long capillary tube.

The following sixty examples were performed on human patients employing the above described methodology and yielded the following results wherein the subject method yielded the following cancer coefficients:

### Example 1

A 49-year-old female was found to have uterine carcinoma (cancer coefficient value of 1.8).

### Example 2

This 44 year old female had a cancer coefficient value of 1.8 with tissue diagnosis of breast carcinoma.

### Example 3

A female adult of unknown age presented with liver metastasis from an adenocarcinoma of unknown origin; her cancer coefficient was 3.6.

### Example 4

A benign testicular tumor was found in this 29 year old male having a cancer coefficient value of zero.

### Example 5

This 66 year old male with prostate cancer had a cancer coefficient value of 4.1.

### Example 6

A cancer coefficient value of 3.8 was found for a 71 year old male with adenocarcinoma of the lung.

### Example 7

A 30 year old female found to have uterine carcinoma with metastases had a cancer coefficient value of 4.1.

### Example 8

A 43 year old female with breast cancer had a cancer coefficient value of 1.6.

### Example 9

Prostate cancer in a 56 year old male was associated with a cancer coefficient value of 6.5.

### Example 10

A 54 year old female with colon cancer and a uterine tumor presented with a cancer coefficient value of 4.3.

### Example 11

This 58 year old female had a cancer coefficient value of 3.6 soon after resection of a bladder transitional cell carcinoma.

### Example 12

An 86 year old female presented with a salivary gland cancer and had a cancer coefficient value of 1.9.

### Example 13

A 66 year old female with lymphosarcoma of the head and neck had a cancer coefficient value of 4.8.

### Example 14

Uterine carcinoma was associated in this 28 year old female with a cancer coefficient value of 7.9.

### Example 15

A 73 year old female with lymphosarcoma had a cancer coefficient value of 9.5.

### Example 16

A 53 year old female with histiocytic lymphocytic leukemia had a cancer coefficient value of 3.8.

### Example 17

A 75 year old male with chronic lymphocytic leukemia had a cancer coefficient value of 3.7.

### Example 18

This 56 year old female with gastric carcinoma had a cancer coefficient value of 2.3.

### Example 19

Esophageal carcinoma in this 66 year old male was associated with a cancer coefficient value of 3.7.

### Example 20

A right upper lobe lung cancer in this 43 year old male was associated with a cancer coefficient value of 2.3.

### Example 21

A 30 year old female who was found to have lymphoma had a cancer coefficient value of 2.8.

### Example 22

A 49 year old male with renal carcinoma had a cancer coefficient value of 10.1.

### Example 23

This 27 year old female had a left ovarian teratocarcinoma and a cancer coefficient value of 3.6.

### Example 24

A 74 year old female found to have colon carcinoma had a cancer coefficient value of 2.2.

### Example 25

A 74 year old male with lymphosarcoma had a cancer coefficient value of 4.8.

### Example 26

This 67 year old male found to have esophageal carcinoma had a cancer coefficient value of 6.2.

### Example 27

A 62 year old male with lymphoma had a cancer coefficient value of 3.5.

### Example 28

A 55 year old female found to have breast cancer had a cancer coefficient value of 1.4.

### Example 29

Ovarian carcinoma was found in this 63 year old female who had a cancer coefficient value of 3.0.

### Example 30

A 66 year old female found to have breast cancer had a cancer coefficient value of 1.4.

### Example 31

A 56 year old female with colon carcinoma had a cancer coefficient value of 4.0.

### Example 32

Gastric carcinoma in this 72 year old female was associated with a cancer coefficient value of 7.4.

### Example 33

This 65 year old male found to have prostate carcinoma had a cancer coefficient value of 3.2.

### Example 34

A 64 year old male with prostate carcinoma had a cancer coefficient value of 1.8.

### Example 35

This 77 year old male with prostate carcinoma had a cancer coefficient value of 1.7.

### Example 36

Lymphosarcoma in this 79 year old male was associated with a cancer coefficient value of 1.9.

### Example 37

A 21 year old male diagnosed with Hodgkin's Lymphoma had a cancer coefficient value of 1.6.

### Example 38

A 56 year old female with lymphoma had a cancer coefficient value of 2.8.

### Example 39

Breast carcinoma in this 45 year old female was associated with a cancer coefficient value of 5.7.

### Example 40

A 43 year old male with stage IIb breast cancer had a cancer coefficient value of 2.2.

### Example 41

This 44 year old male with renal cell carcinoma metastatic to the liver had a cancer coefficient value of 5.5.

### Example 42

A 30 year old patient with lymphoma had a cancer coefficient value of 4.8.

### Example 43

A 24 year old male diagnosed with lung carcinoma had a cancer coefficient value of 2.7.

### Example 44

A 76 year old male diagnosed with lung carcinoma had a cancer coefficient value of 3.1.

### Example 45

Pancreatic carcinoma metastatic to the liver in this 67 year old male was associated with a cancer coefficient value of 4.8.

### Example 46

A 75 year old female was diagnosed with lymphoma had a cancer coefficient value of 2.8.

### Example 47

This 55 year old male with lymphosarcoma had a cancer coefficient value of zero.

### Example 48

A 70 year old male diagnosed with lung cancer had a cancer coefficient value of 0.6.

### Example 49

A 50 year old female with breast carcinoma had a cancer coefficient value of 6.5.

### Example 50

Lymphoma in this 28 year old male was associated with a cancer coefficient value of 0.9.

### Example 51

A dermato-fibrosarcoma diagnosed and surgically resected in this 44 year old female was associated with a post-operative cancer coefficient value of 1.5.

### Example 52

A 67 year old female with a hepato-biliary carcinoma had a cancer coefficient value of 2.3.

### Example 53

This 53 year old male diagnosed with esophageal carcinoma had a cancer coefficient value of 6.4.

### Example 54

A 43 year old female with breast cancer had a cancer coefficient value of 2.2.

### Example 55

A 59 year old female with lymphogranulomatosis had a cancer coefficient value of 1.4.

### Example 56

Lymphoma in this 23 year old male was associated with a cancer coefficient value of zero.

### Example 57

Lung carcinoma in this 46 year old male was associated with a cancer coefficient value of 5.1.

### Example 58

Lymphogranulomatosis in this 31 year old female was associated with a cancer coefficient value of 4.5.

### Example 59

A 36 year old female diagnosed with breast carcinoma had a cancer coefficient value of 5.6.

### Example 60

A 27 year old female diagnosed with lymphosarcoma had a cancer coefficient value of 9.5.

The following twenty three examples were performed on canine patients employing the below described methodology and yielded the following results wherein the subject method yielded the following cancer coefficients:

Glass capillary tubes 50 mm long (Gus-Khrustalniy, Moscow, Russian Federation) were used in all of these exemplary embodiments. For the 50 mm long glass capillary tubes the test and control aliquots of patient blood each comprised 200 μL of patient blood. About 50 ml of pregnant and non-pregnant serums are added to respective patient whole blood aliquots. ESR determinations were taken after one hour. The cancer coefficient normalization factor equals 50 for these glass capillary tubes. All remaining method steps and elements were the same as in the Examples 1-60 presented in the previous paragraphs.

### Example 61

This healthy mixed-breed dog was found to have a cancer coefficient value of 0.2.

### Example 62

An 8 year old Rottweiler with a diagnosis of endometritis but no malignancy had a cancer coefficient value of 0.2.

### Example 63

A stray mixed breed dog having an ovarian malignancy had a cancer coefficient value of 1.8.

### Example 64

A 9 year old Rottweiler having metastatic adenocarcinoma had a cancer coefficient value of 2.0.

### Example 65

A 7 year old dog found to have malignant lesions in the spleen and liver had a cancer coefficient value of 6.0.

### Example 66

This 9 year old poodle with breast cancer had a cancer coefficient value of 3.2.

### Example 67

A mixed breed female dog with breast cancer had a cancer coefficient value of 3.0.

### Example 68

A dog of unknown age found to have metastatic carcinoma with no apparent primary site had a cancer coefficient value of 4.9.

### Example 69

Breast carcinoma was diagnosed in this 13 year old female dog having a cancer coefficient value of 0.8.

### Example 70

A 10 year old female dog having breast carcinoma was found to have a cancer coefficient value of 0.6.

### Example 71

This 13 year old dog with skin carcinoma had a cancer coefficient value of 4.5.

### Example 72

A 3 year old dog having sarcoma with metastases had a cancer coefficient value of 1.7.

### Example 73

A 10 year old female dog diagnosed with breast carcinoma had a cancer coefficient value of 4.0.

### Example 74

Another 10 year old female dog diagnosed with breast carcinoma had a cancer coefficient value of 1.9.

### Example 75

This 11 year old female poodle found to have breast carcinoma had a cancer coefficient value of 2.4.

### Example 76

This 7 year old boxer was diagnosed with fibrosarcoma and had a cancer coefficient value of 4.8.

### Example 77

A healthy non-pregnant 3 year old Rottweiler had a cancer coefficient value of 0.9.

### Example 78

This 10 year old sheepdog had a cancer coefficient value of 0.9.

### Example 79

A healthy non-pregnant 8½ year old female dog had a cancer coefficient value of 1.3.

### Example 80

A 2 year old Spaniel having eye cancer was found to have a cancer coefficient of 1.7.

### Example 81

A healthy non-pregnant 3 year old Rottweiler without cancer had a cancer coefficient value of 1.3.

### Example 82

This 5 year old female dog presenting with metastatic carcinoma had a cancer coefficient value of 2.8.

### Example 83

A 5 year old dog with ear cancer had a cancer coefficient value of 2.2.

For the clinical series of human patients described above the subject cancer screening test methodology demonstrated a sensitivity of 0.88, a specificity of 0.5, and a positive predictive value of 0.98.

### Exemplary Cancer Screening Test Procedure

Two new standardized cuvettes with corresponding capillary tubes are prepared. Two small pipettes, as supplied by SEDIPLAST® (Polymedco, Cortland, N.Y.), are used for blood delivery from patient into cuvettes. An anticoagulant such as heparin or sodium citrate must be added to patient blood, which must be obtained no more than 24 hours before the testing. If cuvettes and capillary tubes made by the manufacturers SEDIPLAST® (Polymedco, Cortland, N.Y.) are used, then no more anticoagulant needs to be added since it has been added to the cuvettes during production. Cuvettes are labeled, such as #1 and #2 and so on.

Then, using the SEDIPLAST® equipment, 400 μL of blood are taken from the green top tube, in which the whole blood is first placed to combine with the heparin contained therein, and transferred into each cuvette: marked #1 and #2. Blood transfer to the cuvettes is done using standardized green top blood delivery tubes containing heparin, which are included with the standardized package of capillary tube equipment. Smaller tubes can be used to take blood from a finger stick. Then,
1. about 50 μL of test serum (A) is added to blood in cuvette #1;
2. about 50 μL of control serum (C) is added to blood in cuvette #2.
3. Each cuvette is shaken by hand or vibrated with a vibrator for about 5-10 seconds.
4. Capillary tubes are placed into each cuvette in a vertical orientation. The blood mixture level meniscus should reach substantially the same height in the capillary tubes and be marked for subsequent reference.
5. After capillary tubes are set up, the ESR process begins. The ESR elapse time may be about 35-40 minutes when using SEDIPLAST® plastic 100 mm long capillary tubes.
6. At the end of the elapse time period, a distance from each borderline between the transparent part of the blood mixture and the colored part of the blood mixture and the meniscus is measured using the scale on the capillary tubes if glass tubes are used or other metric scale for plastic tubes. Two ESR determination values are measured, the maximum ESR “E_{Max}” and the minimum ESR, “E_{Min}” for both capillary tube groupings.
7. If there is no significant difference in ESR borderline heights for the mixtures containing A or C, then the cancer coefficient value from the test will be no greater than 1.5 and the presumptive conclusion is that there is no on-going cancer.
8. If there is a significant difference between the sedimentation heights, e.g., there is a difference between the ESR determinations which yields a cancer coefficient greater than 1.5, then a presumptive conclusion may be made that there may be an on-going cancer, subject to further biopsy test.

When 50 mm long glass capillary tubes (Gus-Khrustalniy, Moscow, Russian Federation) are used, the formula for the cancer coefficient K is:

K=[(E_{Max}−E_{Min})*E_{Max}]/NF, where NF=50 in this case.

When the SEDIPLAST® ESR System (Polymedco, Cortland, N.Y.) 100 mm long capillary tubes are used, the formula for the cancer coefficient K is:

K=(E_{Max}−E_{Min})*E_{Max}/NF, where NF=230.

A cancer coefficient value of 1.5 is considered the high normal value of the cancer coefficient for non-malignant situations. Cancers typically produce higher cancer coefficients, as high as 6-8, and even higher. The probability of a more aggressively malignant cancer is proportionately higher with higher cancer coefficient values. The Applicants are of the opinion that higher cancer coefficient values correlate with the presence of metastatic disease and indicate a more virulent and higher grade malignancy.

When freeze-dried sera are used, the serum is reconstituted to the original concentration using distilled water 1:100 weight/weight (for each 10 grams of freeze-dried sera about one liter of water), or equivalently, 1000 mls of water would be used. If during manufacturing, however, the freeze-dried sera is added to the cuvettes, then additional water should not be added.

Although this invention has been described in connection with specific forms and embodiments thereof, it should be appreciated that various modifications other than those discussed above may be resorted to without departing from the spirit or scope of the invention. For example, equivalent elements may be substituted for those specifically shown and described, certain features may be used independently of other features, and in certain cases, particular locations of elements may be reversed or interposed, all without departing from the spirit or scope of the invention as defined in the appended claims.

What is claimed is:

1. A method of cancer detection in a patient, comprising the steps of:
(a) providing a test serum chosen from the group consisting of serum from a pregnant mammal and fetal embryonic serum from a pre-determined species, respectively;
(b) providing a control serum from a second mammal belonging to said pre-determined species, wherein said second mammal is not pregnant;
(c) providing a pre-determined quantity of whole blood of said patient;
(d) combining a pre-determined quantity of the test serum with said pre-determined quantity of the whole blood of said patient, thereby forming a test mixture,
filling a first capillary tube with said test mixture to a pre-determined level, and
allowing said test mixture in said first capillary tube to separate into a test sedimented erythrocytes column, and a test plasma column on the top thereof;
(e) combining a pre-determined quantity of the control serum with said pre-determined quantity of the whole blood of said patient in a control, thereby forming a control mixture,
filling a second capillary tube with said control mixture to said predetermining level, and
allowing said control mixture in said second capillary tube to separate into a control sedimented erythrocytes column and a control plasma column on the top thereof;
(f) measuring a first height of said test plasma column and a second height of said control plasma column when the difference between said first and second heights reaches a maximal value thereof, thereby establishing a first Erythrocyte Sedimentation Rate (ESR₁) and a second Erythrocyte Sedimentation Rate (ESR₂), respectively;
(g) establishing a maximal measured ESR (“ESRₘₐₓ”) as the largest of said ESR₁ and ESR₂;
(h) determining a cancer coefficient K
K=[(ESR₁−ESR₂)*ESR_{max]/NF,}
wherein NF is a normalization factor depending on an internal diameter of said first and second capillary tubes,
(i) comparing said cancer coefficient K with a pre-determined threshold value; and
(j) identifying said patient as a possible candidate for having an on-going malignant process if said cancer coefficient K exceeds said pre-determined threshold value.

2. The method of cancer detection in a patient as recited in claim 1, wherein said test serum comprises a serum from said pregnant mammal obtained at a predetermined gestational time.

3. The method of cancer detection in a patient as recited in claim 2, wherein said test serum comprises a serum from a pregnant mammal belonging to said pre-determined species during a second trimester.

4. The method of cancer detection in a patient as recited in claim 1, wherein said first capillary tubes and second capillary tubes are tilted from a vertical position thereof in a range of about 10° to about 55°.

5. The method of cancer detection in a patient as recited in claim 1, wherein said first and second capillary tubes are tilted approximately 45° from a vertical position thereof.

6. The method of cancer detection in a patient as recited in claim 1, wherein said test and control mixtures are kept at a temperature in the range of about 20° to about 37° C.

7. The method of cancer detection in a patient as recited in claim 1, wherein said cancer being a malignant neoplastic disease entity chosen from the group of malignant neoplastic disease entities consisting of adenocarcinomas, lymphomas, multiple myelomas, prostate carcinomas, transitional cell bladder carcinomas, squamous cell carcinomas, sarcomas, malignant teratocarcinomas, thyroid carcinomas, pancreatic carcinomas, lung carcinomas, cervical carcinomas, ovarian carcinomas, breast carcinomas, endocrine carcinomas, colon carcinomas, malignant melanomas, testicular cancers, leukemias, gastrointestinal carcinomas, head and neck carcinomas, carcinomas of unknown origin, and combinations thereof.

8. The method of cancer detection in a patient as recited in claim 1, wherein said normalization factor NF=A·D²,
wherein A=34.5, and
D is an internal diameter of said first and second capillary tubes, mm.

9. A method for detecting a malignancy in a patient, comprising:
(a) providing fetal embryonic serum belonging to a pre-determined species;
(b) providing a control serum from a mammal belonging to said pre-determined species, wherein said mammal is not pregnant;
(c) providing a pre-determined quantity of whole blood of the patient;
(d) combining a pre-determined quantity of the fetal embryonic serum with said pre-determined quantity of the whole blood of said patient, thereby forming a test mixture,
filling a first capillary tube with said test mixture to a pre-determined level, and
allowing said test mixture in said first capillary tube to separate into a test sedimented erythrocytes column and a test plasma column on the top of said test sedimented erythrocytes column;
(e) combining said pre-determined quantity of said non-pregnant mammal serum with said pre-determined quantity of the whole blood of said patient, thereby forming a control mixture,
filling a second capillary tube with said control mixture to said predetermining level, and
allowing said control mixture in said second capillary tube to separate into a control sedimented erythrocytes column and a control plasma column on the top of said control sedimented erythrocytes column;
(f) measuring a first height of said test plasma column and a second height of said control plasma column when the difference between said first and second heights reaches a maximal value thereof, thereby establishing a first Erythrocyte Sedimentation Rate (ESR₁) and a second Erythrocyte Sedimentation Rate (ESR₂), respectively;
(g) establishing a maximal measured ESR (“ESRₘₐₓ”) as the largest among said ESR₁ and ESR₁;
(h) determining a cancer coefficient
K=[(ESR₁−ESR₂)*ESR_{max]/NF;}
wherein NF is a normalization factor depending on an inner diameter of said first and second capillary tubes;
(i) comparing said cancer coefficient K with a pre-determined threshold value; and
(j) identifying said patient as a possible candidate for having an on-going malignant process if said calculated cancer coefficient K exceeds said pre-determined threshold value.

10. A method for detecting a malignancy in a patient, comprising the steps of:
(a) providing a test serum chosen from the group consisting of serum from a pregnant mammal and fetal embryonic serum from a pre-determined species;
(b) acquiring a pre-determined quantity of whole blood of said patient;
(c) combining a pre-determined quantity of said test serum with said pre-determined quantity of the whole blood of said patient, thereby forming a test mixture,
filling a first capillary tube with said test mixture to a pre-determined level, and
allowing said test mixture in said first capillary tube to separate into a test plasma column and a test sedimented erythrocytes column;
(d) determining a normalization factor (“NF”),
wherein A=34.5, and NF=A·D²
D is an inner diameter of said first capillary tube;
(e) providing a serum from a non-pregnant mammal belonging to said pre-determined species;
(f) combining a pre-determined quantity of said non-pregnant mammal serum with said pre-determined quantity of the whole blood of said patient, thereby forming a control mixture,
filling a second capillary tube with said control mixture to said predetermining level, and
allowing said control mixture in said second capillary tube to separate into a control plasma column and a control sedimented erythrocytes column;
(g) measuring a first height of said test plasma column and a second height of said control plasma column when the difference between said first and second heights reaches a maximal value thereof, thereby establishing a first Erythrocyte Sedimentation Rate (ESR₁) and a second Erythrocyte Sedimentation Rate (ESR₂), respectively;
(h) establishing a maximal measured ESR (“ESRₘₐₓ”) as the largest among said ESR₁ and ESR₂;
(i) determining a cancer coefficient K
K=[(ESR₁−ESR₂)*ESR_{max]/NF;}
(j) comparing said cancer coefficient with a pre-determined threshold value; and
(k) identifying said patient as a possible candidate for having an on-going malignant process if said calculated cancer coefficient K exceeds said pre-determined threshold value.

11. The method for detecting a malignancy in a patient as recited in claim 10, wherein said first and second capillary tubes are tilted in the range of about 10° to about 55° from a vertical position thereof.

12. The method for detecting a malignancy in a patient as recited in claim 11, wherein said first and second capillary tubes are tilted approximately 45° from a vertical position thereof.

13. The method for detecting a malignancy in a patient as recited in claim 10, wherein said test and control mixtures are kept at a temperature in the range of about 20° to about 37° C.

14. The method for detecting a malignancy in a patient as recited in claim 10, wherein said test serum comprises a serum from said pregnant mammal obtained at a predetermined gestational time

15. The method for detecting a malignancy in a patient as recited in claim 14, wherein said predetermined gestational time of said pregnant mammal is in a second trimester.
